(19) United States
(12) Patent Application Publication
(10) Pub. No.: US 2007/0270874 A1
(43) Pub. Date: Nov. 22, 2007

(54) Surgical distraction device and procedure

(75) Inventor: Kent M. Anderson, Memphis, TN (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 11/409,639

(22) Filed: Apr. 24, 2006

(51) Int. Cl. A61B 17/58

(52) U.S. Cl. 606/90

(57) ABSTRACT

A surgical procedure for distracting two anatomical structures, according to which one or more cams are rotatably mounted on a body member and are inserted between the structures and rotated to distract the structures.

### BACKGROUND

The present invention relates to a surgical distraction device and procedure and, more particularly, to such a device and procedure in which two anatomical structures are distracted.

It is often necessary to distract two anatomical structures as a preliminary step in a surgical procedure. For example, spinal discs that extend between adjacent vertebrae in vertebral columns of the human body provide critical support between the adjacent vertebrae. These discs can rupture, degenerate, and/or protrude due to injury, degradation, disease, or the like, to such a degree that the intervertebral space between adjacent vertebrae collapses as the disc loses at least a part of its support function. This, in turn, can cause impingement of the nerve roots and severe pain.

In these cases, intervertebral prosthetic devices have been designed that can be implanted between the adjacent vertebrae, and more particularly, between the processes of the adjacent vertebrae, to prevent the collapse of the intervertebral space and thus stabilize the spine. However, during these procedures, and prior to implantation of the prosthetic device, it is often difficult to size and distract the space between the spinous process, especially without considerable invasion and possible damage to the processes.

Embodiments of the present invention improve upon these techniques, and various embodiments of the invention may possess one or more of the above features and advantages, or provide one or more solutions to the above problems existing in the prior art.

### BRIEF DESCRIPTION OF THE DRAWINGS

FIG. 1 is a side elevational view of an adult human vertebral column.

FIG. 2 is a posterior elevational view of the column of FIG. 1.

FIG. 3 is an elevational view of one of the vertebrae of the column of FIGS. 1 and 2.

FIG. 4 is an enlarged, partial, isometric view of a portion of the column of FIGS. 1 and 2, including the lower three vertebrae of the column, and showing a distraction device according to an embodiment of the invention.

FIGS. 5A and 5B are enlarged partial elevational views of the distraction device of FIG. 4 showing two operative positions.

FIGS. 6A and 6B are enlarged partial elevational views of an alternate embodiment of the distraction device of FIG. 4 showing two operative positions.

FIGS. 7A and 7B are enlarged partial elevational views of an alternate embodiment of the distraction device of FIG. 4 showing two operative positions.

### DETAILED DESCRIPTION

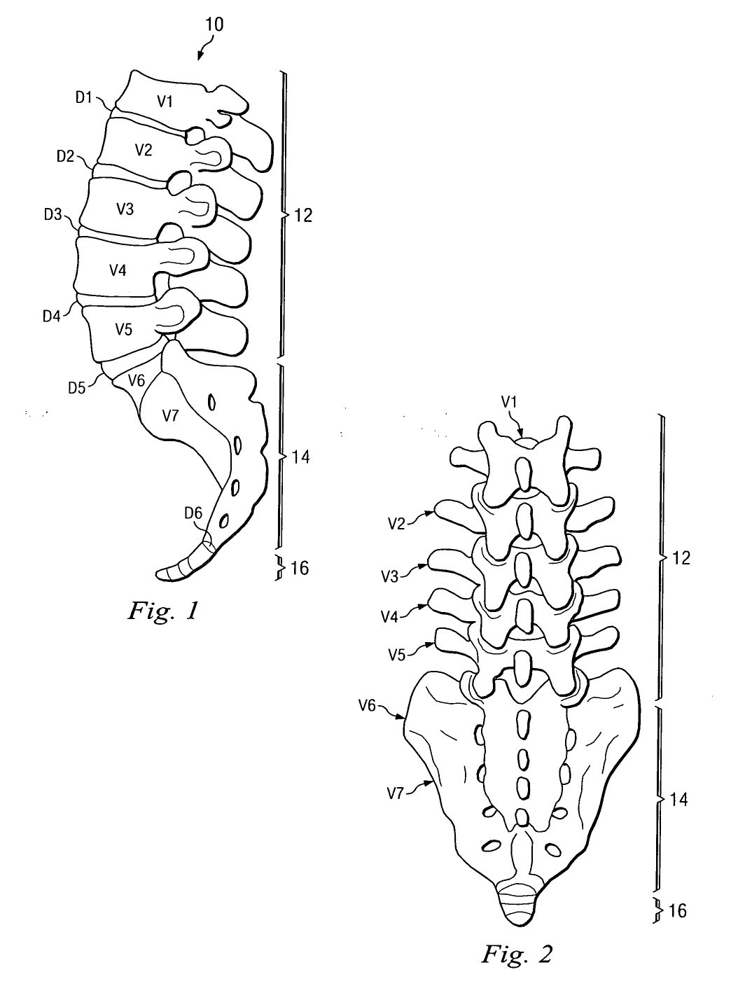

With reference to FIGS. 1 and 2, the reference numeral 10 refers, in general, to the lower portion of a human vertebral column. The column 10 includes a lumbar region 12, a sacrum 14, and a coccyx 16. The flexible, soft portion of the column 10, which includes the thoracic region and the cervical region, is not shown.

The lumbar region 12 of the vertebral column 10 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, the disc D3 extending between the vertebrae V3 and V4, and the disc D4 extending between the vertebrae V4 and V5.

The sacrum 14 includes five fused vertebrae, one of which is a superior vertebra V6 separated from the vertebra V5 by a disc D5. The other four fused vertebrae of the sacrum 14 are referred to collectively as V7. A disc D6 separates the sacrum 14 from the coccyx 16, which includes four fused vertebrae (not referenced).

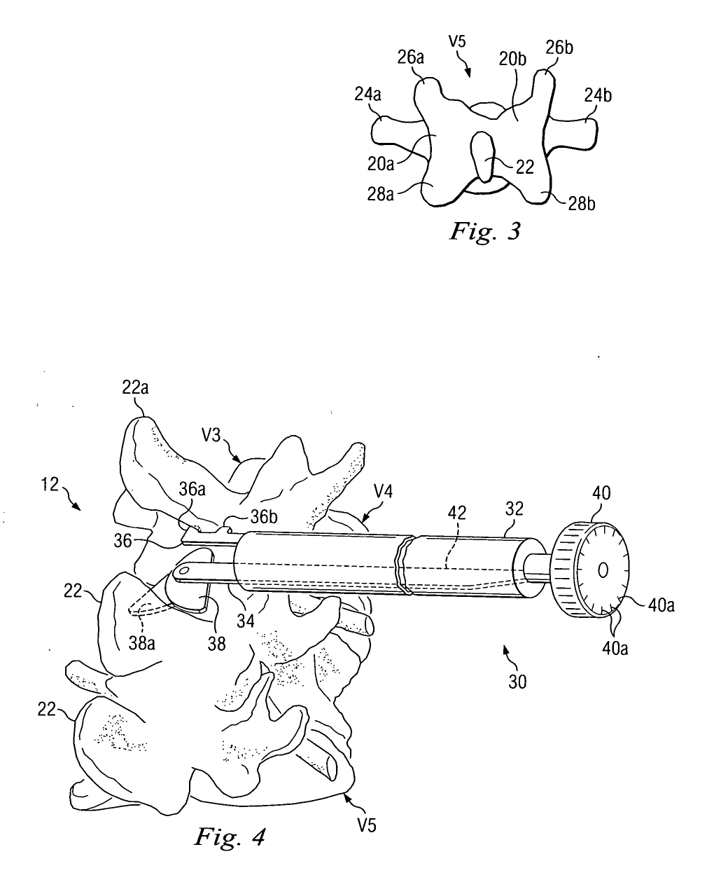

With reference to FIG. 3, the vertebra V5 includes two laminae 20a and 20b extending to either side (as viewed in FIG. 2) of a spinous process 22 that extends posteriorly from the juncture of the two laminae. Two transverse processes 24a and 24b extend laterally from the laminae 20a and 20b, respectively. Two articular processes 26a and 26b extend superiorly from the laminae 20a and 20b, respectively, and two articular processes 28a and 28b extend inferiorly from the laminae 20a and 20b, respectively. The inferior articular processes 28a and 28b rest in the superior articular process of the vertebra V2 to form a facet joint. Since the vertebrae V1-V4 are similar to the vertebra V5, and since the vertebrae V6 and V7 are not involved in the present invention, they will not be described in detail.

It will be assumed that, for one or more of the reasons set forth above, the vertebrae V4 and V5 are not being adequately supported by the disc D4 and that it is therefore necessary to provide supplemental support and stabilization of these vertebrae. To this end, and referring to FIG. 4, a distraction device, shown in general by the reference numeral 30, is positioned between the spinous process 22 of the vertebra V5 and the spinous process of the vertebra V4, with the latter process being referred to by the reference numeral 22a.

The device 30 comprises a cylindrical body member 32 having two arms 34 and 36 projecting from one end thereof. Two cams 38 and 38a are rotatably mounted on the distal end portion of the arm 34 in any conventional manner, and the arm 36 has two spaced dimples 36a and 36b projecting from one surface so as to receive the process 22a. A knob 40 is rotatably mounted in the other end of the body member 32 in a conventional manner, and indicia 40a is provided on the knob 40 that can be aligned with corresponding indicia, or the like (not shown), on the body member to indicate the amount of rotation of the knob, for reasons to be described.

A linkage system 42 is provided in the body member 32 for linking the knob 40 to the cams 38 and 38a. Since the linkage system 42 is conventional, it will not be described or shown in further detail. The arrangement is such that rotation of the knob 40 actuates the linkage system 42 to cause a corresponding rotation of the cams relative to the arm 36.

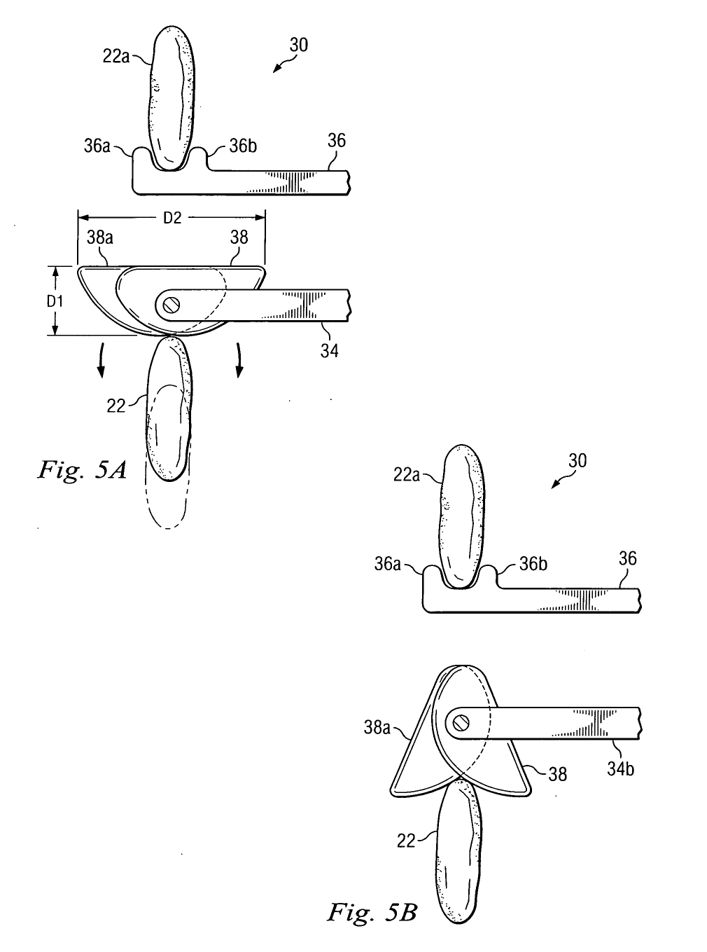

FIG. 5A depicts the arms 34 and 36 and the cams 38 and 38a in greater detail. Each cam 38 and 38a has a straight surface and a curved, semicircular, surface. The dimension D1, corresponding to the width of each cam 38 and 38a, is less than the dimension D2 corresponding to the combined lengths of the cams. The cams 38 and 38a are adjusted so that their respective flat surfaces extend horizontally, with the cam 38a slightly offset from the cam 38 in an axial direction.

According to an exemplary procedure using the device 30, the arms 34 and 36 are inserted between the process 22 and 22a by manually grasping the body member 32 (FIG. 4), and manipulating the device 30 so that the cams 38 and 38a extend close to, or in slight engagement with, the process 22, and so that the nipples 34a and 34b extend to either side of the process 22a. In this position, the axes of the cams 38 and 38a extend substantially perpendicularly to the axis of the process 22, and the relatively small dimension D1 of the cams extends between the processes 22 and 22a. This is the minimum distracted position which has little or no effect on the processes 22 and 22a.

The knob 40 (FIG. 4) can then be rotated by the linkage 42 to rotate, or pivot, the cams 38 and 38a in opposite angular directions for slightly less than ninety degrees until they attain the maximum distracted positions shown in FIG. 5B in which they intersect in one plane and form a “V.” This movement of the cams 38 and 38a forces the process 22 to its maximum distracted position shown in FIG. 5B and by the phantom lines in FIG. 5A. In this position, the upper surface of the process 22 extends in the “V” formed by the cams 38 and 38a. During this movement, the process 22a remains in its position between the nipples 36a and 36b of the arm 36.

It is understood that the cams 38 and 38a can be rotated to, and maintained at, a position between the minimum and maximum distracted positions, as indicated by the position of the indicia 40a (FIG. 4).

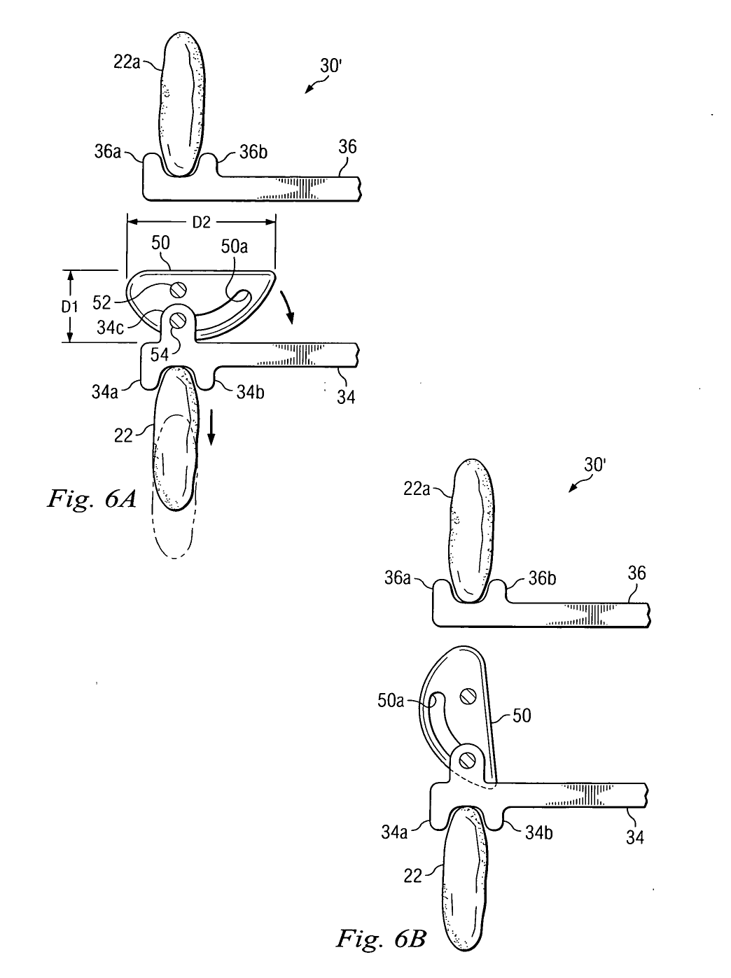

According to the embodiment of FIGS. 6A and 6B, a device 30′ is provided that is similar to the device 30 of the embodiment of FIGS. 5A and 5B, and identical components are given the same reference numerals. In this embodiment, a pair of spaced nipples 34a and 34b are provided on the lower surface of the arm 34 as viewed in the drawing and a flange 34c is mounted on the upper surface. A cam 50 is provided that is shaped similarly to the cam 38 of the previous embodiment and is pivotally mounted about a shaft 52. Although not shown in the drawings in the interest of clarity, it is understood that the cam S0 is supported by the arm in any conventional manner. The cam 50 has a circular slot 50a, and a pin 54 extends through the flange 34c and into the slot. The dimension D1, corresponding to the width of the cam 50, is less than the dimension D2 corresponding to the length of the cam.

According to an exemplary procedure using the device 30′, the cam 50 is adjusted by the linkage 42 (FIG. 4) so that its flat surfaces extends horizontally. The arms 34 and 36 are inserted between the processes 22 and 22a by manually grasping the body member 32 (FIG. 4), and manipulating the device 30′ so that the process 22 extends between the nipples 34a and 34b, and the process 22a extends between the nipples 36a and 36b as shown in FIG. 6A. In this position, the axis of the cam 50 extends substantially perpendicularly to the axis of the process 22. This is the minimum distracted position which has little, or no effect on the processes 22 and 22a.

The knob 40 (FIG. 4) can then be rotated to rotate, or pivot, the cam 50 about the shaft 52 for approximately ninety degrees so that it drives the arm 34 through the engagement of the pin 52 in the slot 50a. The arm thus drives the process 22 to its maximum distracted position shown in FIG. 6B.

It is understood that the cam 50 can be rotated to, and maintained at, a position between the minimum and maximum distracted positions, as indicated by the position of the indicia 40a (FIG. 4). During this movement, the process 22a remains in its position between the nipples 36a and 36b of the arm 36.

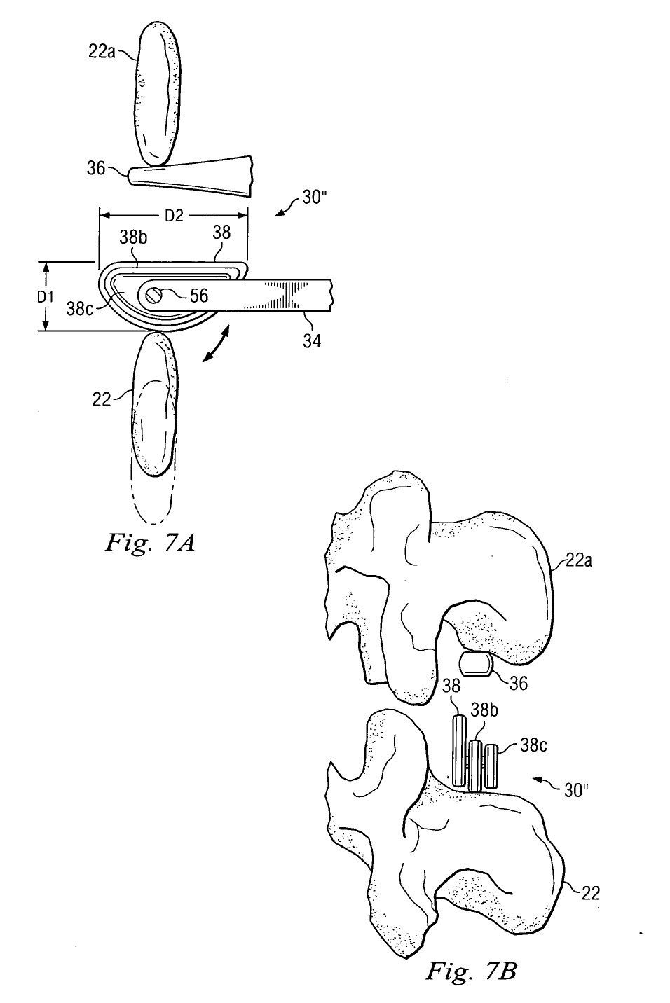

Referring to the embodiment of FIGS. 7A and 7B, a device 30″ is provided that is similar to the device 30 of the embodiment of FIGS. 5A and 5B, and identical components are given the same reference numerals. According to the embodiment of FIGS. 7A and 7B, the cam 38 of the embodiment of FIGS. 5A and 5B is provided along with two additional cams 38b and 38c that are similar to the cam 38 but slightly smaller in size. The width of the cam 38 is less than its length as discussed in the embodiment FIGS. 5A and 5B. Likewise, the width of each cam 38b and 38c is less than its length. The cams 38, 38b and 38c are positioned in a side-by-side relationship and are rotatably mounted about a shaft 56 provided on the arm 34. The cams 38, 38b and 38c can be eccentrically disposed on the shaft, with the degree of eccentricity varying from cam to cam. It is understood that the link 42 can be adapted to actuate all of the cams 38, 38b, and 38c so that rotation of the knob 40 (FIG. 4) causes corresponding rotation of the cams 38, 38b, and 38c.

According to an exemplary procedure using the device 30″, the cams 38, 38b, and 38c are adjusted so that their respective flat surfaces extend horizontally and the arm 34 with the cams, as well as the arm 36, are inserted between the processes 22 and 22a by manually grasping and manipulating the body member 32 (FIG. 4). The device 30″ is positioned so that the arm 36 engages the process 22a and the cams 38, 38b and 38c extend adjacent the process 22.

The knob 40 (FIG. 4) can then be rotated by the linkage 42 (FIG. 4) to move the cams 38, 38b and 38c, and therefore the process 22, to obtain variable distraction, based on the angular position of the cams and the relative angular position between the cams. It is understood that the cams 38, 38b and 38c can be rotated to, and maintained at, a position between a minimum and a maximum distracted position, as needed, as indicated by the position of the indicia 40a on the knob 40.

Although not shown in FIGS. 7 and 8, it is understood that the nipples 36a and 36b of the previous embodiments can be provided on the upper surface of the arm 36. Also, two additional links (not shown), identical to the link 42, can be provided that are respectively connected to two additional knobs (not shown), so that each cam 38, 38b and 38c can be rotated individually by its corresponding knob and link. Further, the cams 38, 38b and 38c can be placed at a predetermined angular position relative to each other before insertion between the process 22 and 22a, and before the knob 40 is used to rotate the cams to obtain distraction based on the angular position of the cams.

In each of the above embodiments, the devices 30, 30′, and 30″ can be used in one of several procedures. For example, each device could be used to distract the structures in the above manner to permit a device, such as a prosthesis, to be implanted between the structures or in an area near the structures, as discussed above. According to another example, each device 30, 30′, and 30″, could be used to distract the structures to permit another surgical procedure to be performed in the space formed by the distraction.

It is also understood that, in each of the above embodiments, either one, or both, of the processes 22 and 22a can be moved during the distraction procedure.

### VARIATIONS

It is understood that variations may be made in the foregoing without departing from the invention and examples of some variations are as follows:

(1) The distraction devices and procedures discussed above can be inserted between anatomical structures other than the two spinous processes disclosed above.

(2) The distraction devices and procedures discussed above can be used in connection with surgical procedures different from the procedures discussed above.

(3) In the embodiments of FIGS. 5A and 5B, as well as 7A and 7B, separate knobs can be provided along with separate links that connect the knobs to the respective cams.

(4) The shapes of the cams can be varied.

(5) In the embodiment of FIGS. 7A and 7B, the number of cams can be varied and one or more can be concentrically mounted on the shaft and one or more eccentrically mounted on the shaft.

(6) The devices disclosed above can be inserted between two vertebrae following a corpectomy in which at least one vertebrae is removed.

(7) The links disclosed above, as well as the manner in which the links are actuated, can vary.

(8) Any spatial references made above, such as “under”, “over”, “between”, “upper”, “lower”, “top”, “bottom”, etc. are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw are equivalent structures.

What is claimed is:

1. A surgical distraction device adapted to distract two anatomical structures comprising:
a body member;
at least one cam rotatably mounted on the body member and having a relatively large dimension and a relatively small dimension, and
means mounted on the body member for rotating the cam,
the cam adapted to be inserted between the structures with its relatively small dimension extending between the structures and rotated towards a position whereby its relatively large dimension extends between the structures to distract the structures.

2. The device of claim 1 wherein the means comprises a knob rotatably mounted on the body member, and a link connecting the knob to the cam so that rotation of the knob causes corresponding rotation of the cam.

3. The device of claim 1 further comprising indicia on the knob for indicating the amount of rotation of the knob and therefore the amount of rotation of the cam.

4. The device of claim 1 wherein the body member comprises two spaced arms one of which engages one of the structures, at least one cam being rotatably mounted on the other arm and engaging the other structure.

5. The device of claim 4 wherein two spaced dimples are mounted on the one arm for receiving the one structure.

6. The device of claim 4 wherein there are two cams rotatably mounted on the other arm and adapted to rotate in opposite directions.

7. The device of claim 5 wherein one cam is axially offset from the other, and each cam engages the corresponding structure during the rotation to distract the structures.

8. The device of claim 1 wherein the body member comprises two spaced arms that respectively engage the structures, and wherein the cam is rotatably mounted on one of the arms.

9. The device of claim 8 wherein two spaced dimples are mounted on each arm for receiving the structures.

10. The device of claim 8 further comprising a pin extending from the one arm and into a slot in the one cam so that rotation of the cam drives the one arm into engagement with its corresponding structure to distract the structures.

11. The device of claim 1 wherein the body member comprises two spaced arms one of which engages one of the structures, and where there are at least two cams rotatably mounted on the other arm and adapted to engage the other structure.

12. The device of claim 11 wherein there are at least three cams rotatably mounted on the other arm.

13. The device of claim 11 wherein the sizes of the cams vary.

14. The device of claim 11 wherein the relatively angular positions of the cams vary.

15. The device of claim 11 further comprising a shaft extending from the arm, at least one of the cams being eccentrically mounted on the shaft with variable degrees of eccentricity.

16. A surgical procedure for distracting two anatomical structures, the procedure comprising:
providing a cam having a relatively large dimension and a relatively small dimension;
rotatably mounting the cam on a body member;
grasping the body member and inserting the cam between the structures with its relatively small dimension extending between the structures; and
rotating the cam relative to the body member towards a position whereby its relatively large dimension extends between the structures to distract the structures.

17. The procedure of claim 16 wherein the step of rotating comprises rotatably mounting a knob on the body member, connecting the knob to the cam, and rotating the knob to cause corresponding rotation of the cam.

18. The procedure of claim 17 further comprising indicating the amount of rotation of the knob and therefore the amount of rotation of the cam.

19. The procedure of claim 16 wherein the step of rotating comprises rotatably mounting two cams on the body member so that they rotate in opposite directions.

20. The procedure of claim 19 further comprising axially offsetting one cam from the other, and engaging each cam with the corresponding structure during the rotation to distract the structures.

21. The procedure of claim 16 wherein the step of rotating comprises driving part of the body member into engagement with its corresponding structure to distract the structures.

22. The procedure of claim 16 wherein there are at least three cams rotatably mounted on the body member and adapted to engage a structure.

23. The procedure of claim 16 further comprising varying the angular position of the cams relative to the body member.

24. The procedure of claim 16 further comprising eccentrically mounting the cams on a shaft of the body member with variable degrees of eccentricity.
